(19) United States
(12) Patent Application Publication
(10) Pub. No.: US 2016/0206641 A1
(43) Pub. Date: Jul. 21, 2016

(54) RAPIDLY-DISSOLVING THIN FILM FORMULATION OF WATER SOLUBLE DIGITALIS GLYCOSIDE FOR THE TREATMENT OF CONGESTIVE HEART DISEASE

(71) Applicant: LUMINUS BIOSCIENCES INC., San Antonio, TX (US)

(72) Inventor: Chandra U. Singh, San Antonio, TX (US)

(21) Appl. No.: 14/912,374

(22) PCT Filed: Aug. 14, 2014
(86) PCT No.: PCT/US14/51142

§ 371 Date: Feb. 16, 2016

Related U.S. Application Data
(60) Provisional application No. 61/866,870, filed on Aug. 16, 2013.

(51) Int. Cl. A61K 31/7048; A61K 9/70; A61K 47/38; A61K 47/40; A61K 47/48; A61K 9/00; A61K 47/36

(52) U.S. Cl. CPC A61K 31/7048; A61K 9/006; A61K 9/7007; A61K 47/36; A61K 47/40; A61K 47/48969; A61K 47/38

(57) ABSTRACT

The present invention provides an orally consumable rapidly dissolving solid film comprising: at least one water soluble polymer, and at least one water soluble digitalis glycoside and optionally at least one amorphous cyclodextrin; and wherein the ratio of the at least one water soluble digitalis glycoside to the optionally at least one amorphous cyclodextrin is about 1:1 to about 1:10 and wherein said orally consumable film is adapted to adhere to and dissolve in the mouth of a subject afflicted with heart disease. The present invention also provides methods of making and using the same.

### CROSS-REFERENCE TO RELATED APPLICATIONS

This application claims the benefit of U.S. Provisional Application No. 61/866,870, filed Aug. 16, 2013, which is incorporated by reference in its entirety.

### BACKGROUND OF THE INVENTION

Ouabain (g-strophanthin) and k-strophanthin are found in the ripe seeds of African plants Strophanthus gratus and the bark of Acokanthera ouabaio. Ouabain is an endogenous hormone. In human plasma from healthy individuals, the circulating levels are normally distributed in the population and range typically from 30-380 pM. Significantly higher levels of endogenous ouabain that may approach or even exceed 1 nM have been observed in many subjects with congestive heart failure, essential hypertension, renal failure and some cancers. It has been suggested that physiological concentrations of ouabain promote cell growth and in some manner stimulate the Na+/K+-ATPase activity while the higher levels achieved during intravenous therapy or in pathophysiological disorders may inhibit the Na+/K+-ATPase (Gao J. et al. Isoform-Specific Stimulation of Cardiac Na/K Pumps by Nanomolar Concentrations of Glycosides. J Gen Physiol. 2002; 119 (4): 297-312).

In France and Germany, intravenous ouabain has a long history in the treatment of heart failure, and some continue to advocate its use intravenously and orally in angina pectoris and myocardial infarction. The positive properties of ouabain regarding the prophylaxis and treatment of these two indications are documented by a clutch of studies (Fürstenwerth H. Ouabain—The Insulin of the Heart. Int J Clin Pract. 2010; 64(12): 1591-4).

Angina pectoris is the result of myocardial ischemia caused by an imbalance between myocardial blood supply and oxygen demand. It is a common presenting symptom (typically, chest pain) among subjects with coronary artery disease (CAD). Approximately 9.8 million Americans are estimated to experience angina annually, with 500,000 new cases of angina occurring every year.

Medical therapies for heart disease are based on a diverse range of drugs. Angiotensin converting enzyme inhibitors, angiotensin II receptor antagonists, β-adrenergic receptor antagonists, aldosterone receptor antagonists, as well as diuretics, and inotropic agents improve clinical symptoms and slow the progression of contractile dysfunction. Despite these therapeutic advances, heart failure is still associated with an annual mortality rate of 10% (Cleland J G F, et al. The effect of cardiac resyncronization on morbidity and mortality in heart failure. N Eng J Med 2005; 352: 1539-49). The search for better treatments and optimization of existing ones remain major challenges in cardiology.

Digoxin and digitoxin are widely used in the treatment of heart diseases. The exact mechanism of action of these drugs has remained an enigma. Ouabain, an endogenous hormone, has become the standard tool to investigate the mode of action of digitalis glycosides, and results with ouabain are regarded as generally valid for all digitalis glycosides. However, there are marked differences between the effects of ouabain and digitalis glycosides. Ouabain has a different therapeutic profile from other digitalis glycosides. Unlike digitalis glycosides, ouabain has a fast onset of action and stimulates myocardial metabolism. The inotropic effect of digitalis glycosides is not related to inhibition of the Na+-K+-ATPase. Ouabain and digitalis glycosides develop their effects in different cellular spaces. Digitalis glycosides increase the intracellular calcium concentration by entering the cell interior and acting on the ryanodine receptors and by forming trans-membrane calcium channels. Ouabain, by activation of the Na+-K+-ATPase from the extracellular side, triggers release of calcium from intracellular stores via signal transduction pathways and activates myocardial metabolism. These data no longer support the concept that all digitalis glycosides exhibit their therapeutic effects by partial inhibition of the ion-pumping function of the Na+-K+-ATPase (Fuerstenwerth H. On the Differences between Ouabain and Digitalis Glycosides. Am J Ther. 2014:21:35-42). Ouabain modulates the metabolism of the heart; it stimulates substrate utilization of the myocardium, removes lactate accumulated during heart diseases and reduces the amount of fatty acids in the blood (Fürstenwerth H. Ouabain—the insulin of the heart. The Int. J. Persp. Clin. Practice., 2010, 64(12): 1591-1594).

Based on decades of extensive clinical experience with ouabain, the therapeutic profile of the drug and the disease profiles for which the use of ouabain is appropriate have been summarized in monographs and reviews (Kern B. Der Myokardinfarkt. 3. Auflage, Heidelberg: Haug Verlag, 1974). The main benefit is in prevention and treatment of acute heart attacks. Prophylactic and therapeutic use of ouabain is recommended in: congestive heart insufficiency without pronounced hypertrophy, coronary sclerosis, cardiogenic hypertension, asthma cardiale, exercise-induced cardiac insufficiency, angina pectoris and arrhythrnias, including those that occur on treatment with digitalis.

The molecular formula of ouabain (FIG. 1) is C₂₉H₄₄O₁₂ and its molecular weight is 584.65. Its chemical name is 1β,3β,5β,11α,14,19-Hexahydroxycard-20(22)-enolide 3-(6-deoxy-α-L-mannopyranoside). The solubility of ouabain in cold water is 10 mg/ml and it is more than 50 mg/ml in hot water. The solubility of digoxin in water is 0.065 mg/mL (20° C.). Thus ouabain is an example of water soluble digitalis glycoside.

In order to enhance the solubility of ouabain, a reversible complex between the ouabain, and a carrier molecule can be formed. The characteristics of the carrier molecule are such that the carrier molecule and the reversible complex are soluble in water. Among these known carrier molecules are cyclodextrin compounds. The use of cyclodextrin derivatives as carriers for pharmaceuticals is reviewed by Albers and Muller (Cyclodextrin derivatives in pharmaceutics. Crit Rev Ther Drug Carrier Syst. 1995: 12:311-37)

A variety of improvements in the characteristics of pharmaceutical complexes including various cyclodextrins and cyclodextrin derivatives are disclosed in the following patents, Noda et al., U.S. Pat. No. 4,024,223 methyl salicylate. Szejtli et al. U.S. Pat. No. 4,228,160 indomethacin. Hyashi et al., U.S. Pat. No. 4,232,009 ω-halo-PGI₂ analogs, Matsumoto et al., U.S. Pat. No. 4,351,846 3-hydroxy and 3-oxo prostaglandin analogs, Yamahira et al., U.S. Pat. No. 4,353,793, bencyclane fumarate. Lipari, U.S. Pat. No. 4,383,992 steroids-corticosteroids, androgens, anabolic steroids, estrogens, progestagens complexed with β-cyclodextrin, but not substituted amorphous β cyclodextrins. Nicolau, U.S. Pat. No. 4,407,795 P-hexadecylaminobenzoic acid sodium salt, Tuttle, U.S. Pat. No. 4,424,209 3,4-diisobutyryloxy-N-[3-(4-isobuttyryloxyphenyl)-1-methyl-n-propyl]-3-phenethylamine, Tuttle, U.S. Pat. No. 4,425,336, 3,4-dihydroxy-N-[3-(4-hydroxyphenyl)-1-methyl-n-propyl]-3-phenethylamine, Wagu et al., U.S. Pat. No. 4,438,106 fatty acids EPA and DHA; Masuda et al., U.S. Pat. No. 4,474,881 2-(2-fluoro-4-biphenyl)propionic acid or salt, Shinoda et al., U.S. Pat. No. 4,478,995 acid addition salt of (2′-benzyloxycarbonyl)phenyl trans-4-guanidinomethylcyclo-hexanecaboxylate, Hyashi et al., U.S. Pat. No. 4,479,944 Prostaglandin 12 analog, Hayashi et al., U.S. Pat. No. 4,479,966, 6,9-methano-prostaglandin I₂ analogs. Harada et al., U.S. Pat. No. 4,497,803 lankacidin-group antibiotic: Masuda U.S. Pat. No. 4,499,085 prostoglandin analog. Szejtli et al., U.S. Pat. No. 4,524,068 piperonyl butoxide, Jones, U.S. Pat. No. 4,555,504 cardiac glycoside, Uekama et al., U.S. Pat. No. 4,565,807 pirprofen, Ueda et al., U.S. Pat. No. 4,575,548 2-nitroxymethyl-6-chloropyridine, Ohwaki et al., U.S. Pat. No. 4,598,070 tripamide anti-hypertensive, Chiesi et al., U.S. Pat. No. 4,603,123 piroxicam (feldene). Hasegawa et al., U.S. Pat. No. 4,608,366 monobenzoxamine, Hiari et al., U.S. Pat. No. 4,659,696 polypeptide. Szejtili et al., U.S. Pat. No. 4,623,641 Prostoglandin 12 methyl ester, Ninger et al., U.S. Pat. No. 4,663,316, unsaturated phosphorous containing antibiotics including phosphotrienin, Fukazawa et al., U.S. Pat. No. 4,675,395 hinokitol. Shimizu et al., U.S. Pat. No. 4,728,509 3-amino-7-isopropyl-5-oxo-5H-[1]-benzopyrano[2,3-b]pyridine-3-carboxcylic acid, Shibani et al. U.S. Pat. No. 4,728,510 milk component, and Karl et al., U.S. Pat. No. 4,751,095 aspartame.

Among the above-mentioned patents, several indicate that complexes of cyclodextrin with drug substances improve the side effect profile of the drug substance. Szejtli et al., U.S. Pat. No. 4,228,160, disclosed that the frequency and severity of gastric and duodenal erosion and ulceration in rats caused by indomethecin is improved in an oral formulation of a complex of β-cyclodextrin and indomethacin in a 2:1 ratio, but is not improved and in fact worsens in the same oral formulation of a complex of 3-cyclodextrin and indomethacin in a 1:1 ratio.

Bodor, U.S. Pat. No. 5,024,998, and Bodor. U.S. Pat. No. 4,983,586, disclose a series of compositions comprising complexes of hydroxypropyl-β-cyclodextrin (IHPCD) complexed to a difficult to solubilize drug, and HPCD complexed to a drug which has first been complexed to a specific class of drug carriers characterized as redox drug carriers. U.S. Pat. No. 5,824,668 discloses the composition of 5β steroid with cyclodextrin suitable for parenteral administration for treating various diseases.

Muller et al. (Complex formation of β- and γ-cyclodextrins with digitoxin, Acta Pharm. Nord. 1992; 4: 313-317) describes the complex formation of digitoxin with β- and γ-cyclodextrins. Uekama et al. (Improvement of the oral bioavailability of digitalis glycosides by cyclodextrin complexation. J Pharm Sci 1983; 72: 1338-41) describes the inclusion complexes of the digitalis glycosides digitoxin, digoxin, and methyl digoxin with three cyclodextrins, the α, β, and γ homologues, in water and in the solid state were studied by a solubility method, IR and ¹H-NMR spectroscopy, and X-ray diffractometry. Solid complexes in a molar ratio of 1:4 of the digitalis glycosides with γ-cyclodextrin were prepared and their in-vivo absorption examined. The rapidly dissolving form of the γ-cyclodextrin complex significantly increased plasma levels of digoxin (approximately 5.4-fold) after oral administration to dogs. Ueda et al. (Interaction of cyclomaltononaose (delta-CD) with several drugs. Drug Dev Ind Pharm 1999; 25: 951-4) examined the complex formation of digitoxin with δ-cyclodextrin and observed enhanced solubility.

U.S. Pat. No. 6,407,079 discloses the pharmaceutical compositions comprising inclusion compounds of sparingly water soluble or water labile drugs with 1-cyclodextrin ethers or 3-cyclodextrin esters and the process for the preparation of such compositions. The patent claims cardiac glycosides as one of the types drugs for the treatment of cardiac disorders. The patent further states that molar ratio of the drug to the cyclodextrin derivative is from about 1:6 to 4:1.

Oral delivery thin-film strips are designed to wet and dissolve quickly upon contact with saliva and buccal tissue, therefore releasing the contained pharmaceutical components (Radhakisan U R, et al. Mouth Dissolving Film and their Patent: An Overview, IR.JP 2012, 3 (9)). The main component of these thin films is one or more hydrophilic polymers, some of which have good mucoadhesive properties. In such case, the polymeric thin film strongly adheres to buccal tissue until complete dissolution. Rapid dissolution and mucoadhesion are key properties important for subject compliance and improved administration of the contained therapeutics. These thin-film strips provide a convenient way to deliver pharmaceutical components (i.e. acetaminophen, dental care products and breath refresher).

For example, WO 99/17753 discloses rapidly dissolving films for delivery of drugs to be adsorbed in the digestive tract. WO 98/26780 discloses a flat, foil, paper or wafer type presentation for the application and release of active substances in the buccal cavity. The specific active ingredient disclosed in WO 98/26780 is buprenorphine.

WO 98/20862 discloses a film for use in the oral cavity that can contain a cosmetic or pharmaceutical active substance.

WO 98/26763 discloses a flat, foil, paper or wafer like presentation for release of active substances into the buccal cavity. The particular active disclosed is apomorphine.

U.S. patent application Ser. No. 09/395,104 also discloses the delivery of pharmaceutical agents in an edible film vehicle.

U.S. Pat. No. 5,411,945 to Ozaki et al. discloses a pullulan binder and products produced therewith, including edible films (Example B-2). The products can include a variety of ingredients in addition to pullulan, such as other polysaccharides, antibacterial agents, flavor-imparting agents and pharmaceutically active substances.

U.S. Pat. No. 3,784,390 to Hijiya et al. discloses pullulan films and their use in coating and packing materials for foods, pharmaceuticals and other oxygen sensitive materials. All of the examples in this patent teach mixing pullulan in hot water.

U.S. Pat. No. 7,067,116 discloses physiologically acceptable films, including edible films. The films include a water soluble film-forming polymer, such as pullulan, and a taste masked pharmaceutically active agent, such as dextromethorphan. The taste masking agent is preferably a sulfonated polymer ion exchange resin comprising polystyrene cross-linked with divinylbenzene, such as AMBERLITE. Methods for producing the films are also disclosed.

U.S. Pat. No. 7,049,479 discloses ultra-thin film transdermal/dermal or transmucosal/mucosal delivery system. U.S. Pat. No. 7,425,292 discloses thin film with non-self-aggregating uniform heterogeneity and drug delivery systems made there from. U.S. Pat. No. 7,442,849 discloses thin film delivery system and method of manufacture. U.S. Patent application 20110305768 discloses quick-dissolving oral thin film for targeted delivery of therapeutic agents. U.S. Patent application 20120128848 discloses thin film with non-self-aggregating uniform heterogeneity and drug delivery systems made therefrom. U.S. Patent application 20060210610 discloses Methods for modulating dissolution, bioavailability, bioequivalence and drug delivery profile of thin film drug delivery systems, controlled-release thin film dosage formats, and methods for their manufacture and use.

The inventor is not aware of any suggestion in the published art that cyclodextrins can act as soluble enhancers in a fast or rapidly dissolving orally consumable film. Accordingly, an object of this invention is to provide fast dissolving orally consumable films containing a cyclodextrin to enhance the solubility of a water soluble digitalis glycoside therein.

The present invention addresses the rapidly-dissolving orally consumable thin film formulation comprising water soluble digitalis glycosides and optionally a solubility enhancer for oral administration. In particular, the present invention addresses the rapidly-dissolving orally consumable thin film comprising ouabain, k-strophanthin or mixtures thereof and optionally an amorphous cyclodextrin for treating heart disease.

### SUMMARY OF THE INVENTION

The present invention relates to the rapidly-dissolving orally consumable thin film formulations of water soluble digitalis glycosides such as ouabain. The water solubility of these digitalis glycosides is enhanced by suitable cyclodextrins. In certain preferred embodiments, the invention relates to the use of the rapidly dissolving thin film comprising ouabain for the prevention and treatment of heart disease.

In another embodiments, the invention relates to prophylactic and therapeutic use of the rapidly-dissolving orally consumable thin film comprising ouabain for treating the conditions: congestive heart insufficiency without pronounced hypertrophy, coronary sclerosis, cardiogenic hypertension, cardiac asthma, exercise-induced cardiac insufficiency, angina pectoris and arrhythmias, including those that occur on treatment with digitalis.

Another aspect of the present invention relates to a method for the treatment of congestive heart insufficiency without pronounced hypertrophy, coronary sclerosis, cardiogenic hypertension, cardiac asthma, exercise-induced cardiac insufficiency, angina pectoris and arrhythmias, including those that occur on treatment with digitalis, in a subject, the method comprising administering orally the rapidly dissolving thin film comprising ouabain to the subject an effective amount. The composition may further comprise a cyclodextrin, preferably an amorphous cyclodextrin.

In certain preferred embodiments, the invention provides a consumable film adapted to adhere to and dissolve in a mouth of a subject, wherein the film comprises at least one water soluble polymer, at least water soluble digitalis glycoside and optionally at least one amorphous cyclodextrin.

In certain embodiments, a method is provided for preparing the consumable film of the invention, comprising: dissolving water-soluble ingredients in water to provide an aqueous solution; mixing at least one water soluble film former and at least one stabilizing agent to provide a film-forming mixture; combining the film-forming mixture and the aqueous solution to provide a hydrated polymer gel; mixing oils to form an oil mixture; adding the oil mixture to the hydrated polymer gel and mixing to provide a uniform gel; casting the uniform gel on a substrate; and drying the cast gel to provide the film.

In the preferred embodiments, the water soluble digitalis glycoside is selected from ouabain, k-strophanthin, k-strophanthidin or mixtures thereof.

In certain embodiments, wherein the composition comprises from 2% to 10% by weight of the digitalis glycoside. In certain embodiments, the amorphous cyclodextrin has a degree of substitution of 2 to 7. In certain embodiments, the ratio by weight of digitalis glycoside to amorphous cyclodextrin is 0.5 to 10.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Chemical Structure of Ouabain and K-Strophanthin

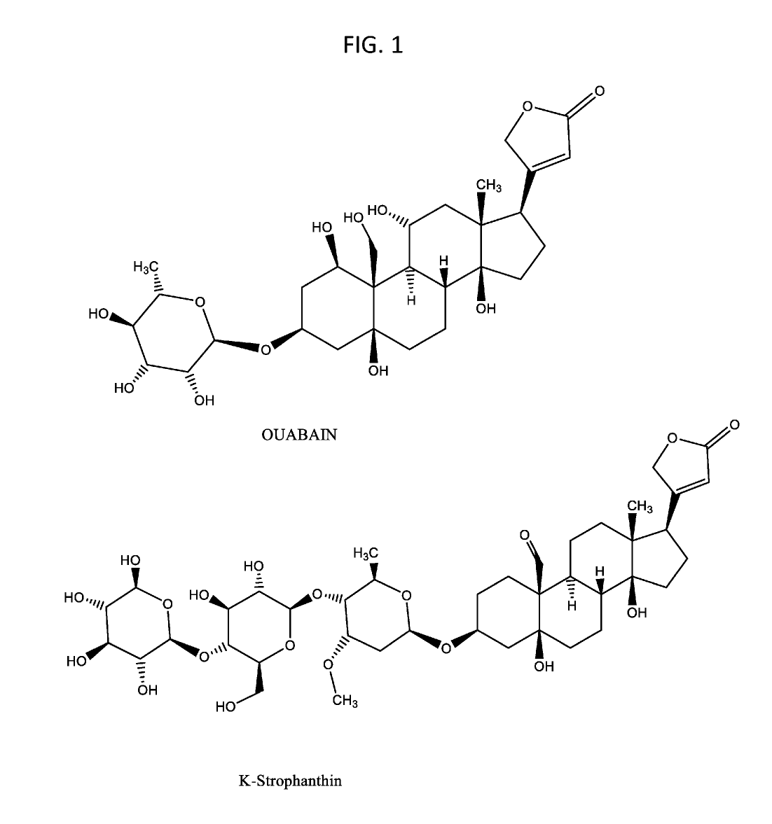

### DETAILED DESCRIPTION OF THE INVENTION

### Definitions

It is understood that the “digitalis activity” of a molecule refers to the ability of the molecule to inhibit Na+/K+-ATPase through acting on the digitalis receptor, along with the ability to induce a positive inotropic effect. Such activity is observed in several natural, semisynthetic and synthetic compounds (Thomas R E: In Molecular Structure and Biological Activity of Steroids; Bohl M, Daux W L. Eds.; CRC Press: Boca Raton, 1992; pp 399-464). Among the natural compounds, there are three groups; steroidal butenolides and pentadienolides, known as “cardiotonic steroids” or “digitalis glycosides” and Erythrophleumalkaloids. The word “digitalis” is often used as a generic word for all cardiotonic steroids. Similarly, the receptor for these compounds is generally known as the “digitalis receptor”. Digitalis glycosides are also called cardiac glycosides and are compounds bearing a steroidal genin or aglycone with one or several sugar molecules attached to position C₃. In the case of the toad venoms, the sugar is replaced by a suberylarginine moeity.

The term “water soluble digitalis glycoside” refers to a digitalis glycoside which is soluble in water. Examples of such glycosides are ouabain and k-strophanthin. Digitalis glycosides increase the intracellular calcium concentration by entering the cell interior and acting on the ryanodine receptors and by forming transmembrane calcium channels. On the other hand, water soluble digitalis glycosides such as ouabain, by activation of the Na+-K+-ATPase from the extracellular side, triggers release of calcium from intracellular stores via signal transduction pathways and activates myocardial metabolism. Further, the water soluble digitalis glycoside such as ouabain modulates the metabolism of the heart; it stimulates substrate utilization of the myocardium, removes lactate accumulated during heart diseases and reduces the amount of fatty acids in the blood. For the purpose of teaching the present invention, any digitalis glycoside whose solubility in water at 25° C. is at least 5 mg/ml shall be called water soluble digitalis glycoside.

As used herein, the term “micron” refers to a unit of measure of one one-thousandth of a millimeter.

As used herein, the term “nm” or the term “nanometer” refers to a unit of measure of one one-billionth of a meter.

As used herein, the term “ng” or the term “nanogram” refers to a unit of measure of one one-billionth of a gram.

As used herein, the term “μg” or the term “microgram” refers to a unit of measure of one one-millionth of a gram.

As used herein, the term “ml” refers to a unit of measure of one one-thousandth of a liter.

As used herein, the term “mM” refers to a unit of measure of one one-thousandth of a mole.

As used herein, the term “μM” refers to a unit of measure of one one-millionth of a mole.

As used herein, the term “nM” refers to a unit of measure of one one-billionth of a mole.

As used herein, the term “pM” refers to a unit of measure of one one-trillionth of a mole.

By “cyclodextrin” is meant α, β, or γ-cyclodextrin. Cyclodextrins are described in detail by Pitha et al., in U.S. Pat. No. 4,727,064, which is incorporated herein by reference. Cyclodextrins are cyclic oligomers of glucose. These compounds form inclusion complexes with many molecules that can fit into and be reversibly bound within the lipophilic cavity of the cyclodextrin molecule.

By “amorphous cyclodextrin” is meant non-crystalline mixtures of cyclodextrins wherein the mixture is prepared from α, β, or γ-cyclodextrin or any derivatives thereof both natural and synthetic. In general the amorphous cyclodextrin is prepared by non-selective additions, especially alkylation of the desired cyclodextrin species. Reactions are carried out to yield mixtures containing a plurality of components thereby preventing crystallization of the cyclodextrin. Various alkylated and hydroxyalkyl-cyclodextrins can be made and of course will vary, depending upon the starting species of cyclodextrin and the addition agent used. Among the amorphous cyclodextrins suitable for compositions according to the invention are hydroxypropyl, hydroxyethyl, glucosyl, maltosyl and maltotriosyl derivatives of 1-cyclodextrin, carboxyamidomethyl-β-cyclodextrin, carboxymethyl-β-cyclodextrin, hydroxypropyl-β-cyclodextrin and diethylamino-β-cyclodextrin. The substituted γ-cyclodextrins may also be suitable, including but not necessarily limited to hydroxypropyl, hydroxyethyl, glucosyl, maltosyl and maltotriosyl derivatives of γ-cyclodextrin. In the compositions according to the invention hydroxypropyl-β-cyclodextrin and hydroxypropyl-γ-cyclodextrin is preferred.

The terms “mixture,” “mix.” and “mixing” or any variants of these terms, when used in the claims and/or specification includes, stirring, blending, dispersing, milling, homogenizing, and other similar methods. The mixing of the components or ingredients of the disclosed compositions can form into a solution. In other embodiments, the mixtures may not form a solution. The ingredients/components can also exist as undissolved colloidal suspensions.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive.

The term “about” is used to indicate that a value includes the inherent variation of error for the device, the method being employed to determine the value, or the variation that exists among the study subjects. It can also refer to ±10% of a specified amount. For example about 100 mean 100±10 of a given quantity.

The expression “physiologically acceptable” as used herein is intended to encompass compounds, which upon administration to a subject, are adequately tolerated without causing undue negative side effects. The expression encompasses edible compounds.

The term “Optional” or “optionally” means that the subsequently described circumstance may or may not occur, so the description includes instances where the circumstance occurs and instances where it does not. For example, recitation of an additive as “optionally present” in a formulation herein encompasses both the formulation containing the additive and the formulation not containing the additive.

The term “rapidly-dissolving or fast dissolving” thin film refers to the oral thin film dosage form which dissolves in the mouth within 10-25 seconds without the need of water.

The expression “pharmaceutically active agents” as used herein is intended to encompass agents other than foods, which promote a structural and/or functional change in and/or on bodies to which they have been administered. These agents are not particularly limited; however, they should be physiologically acceptable and compatible with the film.

The water soluble digitalis glycosides of the present invention, optionally in a cyclodextrin complex, may be in the form of pharmaceutically acceptable salts, esters, amides or prodrugs or combinations thereof. However, conversion of inactive ester, amide or prodrug forms to an active form must occur prior to or upon reaching the target tissue or cell. Salts, esters, amides and prodrugs of the active agents may be prepared using standard procedures known to those skilled in the art of synthetic organic chemistry and described, for example, by J. March, Advanced Organic Chemistry: Reaction, Mechanisms and Structure, 4th Ed. (New York: Wiley-Interscience, 1992).

Certain preferred embodiments of the present invention involve the use of compositions comprising at least one water soluble digitalis glycoside and at least one cyclodextrin or cyclodextrin derivative. As stated above, cyclodextrin or cyclodextrin derivatives may be used as carrier molecules or solubility enhancers. The present invention contemplates the use of cyclodextrin to complex and thereby enhance the solubility of the water soluble digitalis glycoside for administration using rapidly-dissolving thin film to a subject to treat a disease. The cyclodextrin of the compositions according to the invention may be α, β or γ cyclodextrin. α-Cyclodextrin contains six or more glucopyranose units; β-cyclodextrin contains seven glucopyranose units, and γ-cyclodextrin contains eight glucopyranose units. The molecules are believed to exist as truncated cones having a core openings of 4.7 to 5.3 Å, 6.0 to 6.5 Å and 7.5 to 8.3 Å for α, β, or γ-cyclodextrin respectively. The composition according to the invention may comprise a mixture of two or more of the α, β, or γ-cyclodextrins. Usually, however, the composition according to the invention will comprise only one of the α, β, or γ-cyclodextrins. The particular α, β, or γ-cyclodextrin to be used with the particular water soluble digitalis glycosides such as ouabain and k-strophanthin to form the compositions according to the invention may be selected based on the known size of the molecule of the digitalis type of cardiac glycosides such as oleandrin, digitoxin, digoxin and the relative size of the cavity of the cyclodextrin compound and its corresponding complexation affinity.

Generally if the molecule of the water soluble digitalis glycosides such as ouabain is relatively large, a cyclodextrin having a larger cavity is used to make the composition according to the invention. The unmodified α, β, or γ cyclodextrins are less preferred in the compositions according to the invention because the unmodified forms tend to crystallize and are relatively less soluble in aqueous solutions. More preferred for the compositions according to the invention are the α, β, and γ-cyclodextrins that are chemically modified or substituted. Chemical substitution at the 2, 3 and 6 hydroxyl groups of the glucopyranose units of the cyclodextrin rings can yield increases in solubility of the cyclodextrin compound.

Most preferred cyclodextrins in the compositions according to the invention are amorphous cyclodextrin compounds. Amorphous cyclodextrins are non-crystalline mixtures of cyclodextrins wherein the mixture is prepared from α, β, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetamide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl derivatives of β-cyclodextrin carboxyamidomethyl-β-cyclodextrin, carboxymethyl-β-cyclodextrin, hydroxypropyl-β-cyclodextrin and diethylamino-β-cyclodextrin. In the compositions of the present invention, hydroxypropyl-β-cyclodextrin and hydroxypropyl-γ-cyclodextrin is preferred although a analogs may also be suitable. The particular alkylated α, β or γ-cyclodextrin to be used with the particular compound of water soluble digitalis glycosides such as ouabain and k-strophanthin to form the compositions according to the invention will be selected based on the size of the molecule of the compound and the relative size of the cavity of the cyclodextrin compound. As with the unsubstituted cyclodextrins mentioned above, it may be advantageous to use an alkylated cyclodextrin having a larger cavity when the composition according to the invention also includes an excipient. The use of a particular α, β, or γ-cyclodextrin with a particular water soluble digitalis glycosides and excipient in the compositions of the present invention may of course be optimized based on the effectiveness in maintaining the cardiac glycoside in solution.

In certain preferred embodiments, an aqueous preparation of preferably substituted amorphous cyclodextrin and one or more water soluble digitalis glycosides may be prepared. The relative amounts of water soluble digitalis glycosides and cyclodextrin will vary depending upon the relative amount of each of the digitalis glycosides and the effect of the cyclodextrin on the compound. In general, the ratio of the weight of compound of the water soluble digitalis glycosides to the weight of cyclodextrin compound will be in a range between about 1:1 and about 1:100. A weight to weight ratio in a range of about 1:10 to about 1:50 and more preferably in a range of about 1:4 to about 1:10 of a water soluble digitalis glycoside to cyclodextrin is believed to be most effective for increased availability of the digitalis glycoside. For example, ouabain, k-strophanthin or mixtures thereof in a ratio of between about 1:4 and about 1:10 drug to amorphous cyclodextrin, wt/wt, and a final concentration of about 3-6 mg in a strip is expected to significantly enhance the bioavailability as compared to free drug due to the complexation with amorphous cyclodextrin.

Amorphous hydroxypropyl-β- and γ-cyclodextrins may be purchased or synthesized. Amorphous hydroxypropyl-β-cyclodextrin and hydroxypropyl-γ-cyclodextrin may be purchased from a number of vendors including Sigma-Aldrich, Inc. (St. Louis, Mo., USA). In addition, other forms of amorphous cyclodextrin having different degrees of substitution or glucose residue numbers are available commercially. A method for the production of hydroxypropyl-β-cyclodextrin is disclosed in Pitha et al., in U.S. Pat. No. 4,727,064, which is incorporated herein by reference.

The invention provides a physiologically acceptable film that is particularly well adapted to adhere to and dissolve in a mouth of a subject to deliver a water soluble digitalis glycoside. Preferred films according to the invention comprise ouabain, k-strophanthin or a mixture thereof, optionally an amorphous cyclodextrin, a film-forming agent, and at least one of the following additional ingredients: water, antimicrobial agents, plasticizing agents, flavoring agents, saliva stimulating agents, cooling agents, surfactants, stabilizing agents, emulsifying agents, thickening agents, binding agents, coloring agents, sweeteners, fragrances, triglycerides, preservatives, polyethylene oxides, propylene glycol, and the like.

The amount of pharmaceutically active agent that can be used in the rapidly dissolving films, according to the present invention, is dependent upon the dose needed to provide an effective amount of the pharmaceutically active agent. Examples of doses for ouabain, k-strophanthin or mixtures thereof, that can be delivered per one strip of rapidly dissolving oral film is between 1 mg and 10 mg.

The film-forming agent used in the films according to the present invention can be selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. A preferred film former is pullulan, in amounts ranging from about 0.01 to about 99 wt %, preferably about 30 to about 80 wt %, more preferably from about 45 to about 70 wt % of the film and even more preferably from about 60 to about 65 wt % of the film.

Unless specified otherwise, the term “wt %” as used herein with reference to the final product (i.e., the film, as opposed to the formulation used to create it), denotes the percentage of the total dry weight contributed by the subject ingredient. This theoretical value can differ from the experimental value, because in practice, the film typically retains some of the water and/or ethanol used in preparation.

In embodiments containing relatively high oil content, it is preferable to avoid substantial amounts of humectant in the film (and more preferable to have no humectant in the film), so as to avoid producing an overly moist, self-adhering film. In particular, it is preferred to formulate high oil content films with a plasticizing agent other than glycerin, which is also a humectant, and with a sweetener other than sorbitol, which is a mild humectant.

Saliva stimulating agents can also be added to the films according to the present invention. Useful saliva stimulating agents are those disclosed in U.S. Pat. No. 4,820,506. Saliva stimulating agents include food acids such as citric, lactic, malic, succinic, ascorbic, adipic, fumaric and tartaric acids. Preferred food acids are citric, malic and ascorbic acids. The amount of saliva stimulating agents in the film is from about 0.01 to about 12 wt %, preferably about 1 wt % to about 10 wt %, even more preferably about 2.5 wt % to about 6 wt %.

Preferred plasticizing agents include triacetin in amounts ranging from about 0 to about 20 wt %, preferably about 0 to about 2 wt %. Other suitable plasticizing agents include monoacetin and diacetin.

Preferred cooling agents include monomenthyl succinate, in amounts ranging from about 0.001 to about 2.0 wt %, preferably about 0.2 to about 0.4 wt %. A monomenthyl succinate containing cooling agent is available from Mane, Inc. Other suitable cooling agents include WS3, WS23, Ultracool II and the like.

Preferred surfactants include mono and diglycerides of fatty acids and polyoxyethylene sorbitol esters, such as, Atmos 300 and Polysorbate 80. The surfactant can be added in amounts ranging from about 0.5 to about 15 wt %, preferably about 1 to about 5 wt % of the film. Other suitable surfactants include pluronic acid, sodium lauryl sulfate, and the like.

Preferred stabilizing agents include xanthan gum, locust bean gum and carrageenan, in amounts ranging from about 0 to about 10 wt %, preferably about 0.1 to about 2 wt % of the film. Other suitable stabilizing agents include guar gum and the like.

Preferred emulsifying agents include triethanolamine stearate, quaternary ammonium compounds, acacia, gelatin, lecithin, bentonite, veegum, and the like, in amounts ranging from about 0 to about 5 wt %, preferably about 0.01 to about 0.7 wt % of the film.

Preferred thickening agents include methylcellulose, carboxyl methylcellulose, and the like, in amounts ranging from about 0 to about 20 wt %, preferably about 0.01 to about 5 wt %.

Preferred binding agents include starch, in amounts ranging from about 0 to about 10 wt %, preferably about 0.01 to about 2 wt % of the film.

Suitable sweeteners that can be included are those well known in the art, including both natural and artificial sweeteners. Suitable sweeteners include, e.g.:

A. water-soluble sweetening agents such as monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin;
B. water-soluble artificial sweeteners such as the soluble saccharin salts. i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;
C. dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexyen)-alanine, and the like;
D. water-soluble sweeteners derived from naturally occurring water-soluble sweeteners, such as a chlorinated derivative of ordinary sugar (sucrose), known, for example, under the product description of sucralose; and
E. protein based sweeteners such as thaumatoccous danielli (Thaumatin I and II).

In general, an effective amount of auxiliary sweetener is utilized to provide the level of sweetness desired for a particular composition, and this amount will vary with the sweetener selected. This amount will normally be 0.01% to about 10% by weight of the composition when using an easily extractable sweetener. The water-soluble sweeteners described in category A above, are usually used in amounts of about 0.01 to about 10 wt %, and preferably in amounts of about 2 to about 5 wt %. Some of the sweeteners in category A (e.g., glycyrrhizin) can be used in amounts set forth for categories B E below due to the sweeteners' known sweetening ability. In contrast, the sweeteners described in categories B E are generally used in amounts of about 0.01 to about 10 wt %, with about 2 to about 8 wt % being preferred and about 3 to about 6 wt % being most preferred. These amounts may be used to achieve a desired level of sweetness independent from the flavor level achieved from any optional flavor oils used. Of course, sweeteners need not be added to films intended for non-oral administration.

The flavorings that can be used include those known to the skilled artisan, such as natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. These flavorings can be used individually or in admixture. Commonly used flavors include mints such as peppermint, artificial vanilla, cinnamon derivatives, and various fruit flavors, whether employed individually or in admixture. Flavorings such as aldehydes and esters including cinnamyl acetate, cinnamaldehyde, citral diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63 258, may be used. Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine. i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof: and the like.

The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. In general, amounts of about 0.1 to about 30 wt % are useable with amounts of about 2 to about 25 wt % being preferred and amounts from about 8 to about 10 wt % are more preferred.

The compositions of this invention can also contain coloring agents or colorants. The coloring agents are used in amounts effective to produce the desired color. The coloring agents useful in the present invention, include pigments such as titanium dioxide, which may be incorporated in amounts of up to about 5 wt %, and preferably less than about 1 wt %. Colorants can also include natural food colors and dyes suitable for food, drug and cosmetic applications. These colorants are known as FD&C dyes and lakes. The materials acceptable for the foregoing spectrum of use are preferably water-soluble, and include FD&C Blue No. 2, which is the disodium salt of 5,5-indigotindisulfonic acid. Similarly, the dye known as Green No. 3 comprises a triphenylmethane dye and is the monosodium salt of 4-[4-N-ethyl-p-sulfobenzylamino) diphenyl-methylene]-[1-N-ethyl-N-p-sulfonium benzyl)-2,5-cyclo-hexadienimine]. A full recitation of all FD&C and D&C dyes and their corresponding chemical structures may be found in the Kirk-Othmer Encyclopedia of Chemical Technology, Volume 5. Pages 857 884, which text is accordingly incorporated herein by reference.

The films can also include a triglyceride. Examples of triglycerides include vegetable oils such as corn oil, sunflower oil, peanut oil, olive oil, canola oil, soybean oil and mixtures thereof. A preferred triglyceride is olive oil. The triglyceride is added to the film in amounts from about 0.1 wt % to about 12 wt %, preferably in a range from about 0.5 wt % to about 9 wt %, of the film.

The films can include a preservative in amounts from about 0.001 wt % to about 5 wt %, preferably from about 0.01 wt % to about 1 wt % of the film. Preferred preservatives include sodium benzoate and potassium sorbate. Other suitable preservatives include, but are not limited to, salts of edetate (also known as salts of ethylenediaminetetraacetic acid, or EDTA, such as disodium EDTA) and parabens (e.g., methyl, ethyl, propyl or butyl-hydroxybenzoates, etc.) or sorbic acid. The preservatives listed above are exemplary, but each preservative must be evaluated on an empirical basis, in each formulation, to assure the compatibility and efficacy of the preservative. Methods for evaluating the efficacy of preservatives in pharmaceutical formulations are known to those skilled in the art.

The films can also include a polyethylene oxide compound. The molecular weight of the polyethylene oxide compound ranges from about 50,000 to about 6,000,000. A preferred polyethylene oxide compound is N-10 available from Union Carbide Corporation. The polyethylene oxide compound is added in amounts from about 0.1 wt % to about 5 wt %, preferably from about 0.2 wt % to about 4.0 wt % of the film.

The films can also include propylene glycol. The propylene glycol is added in amounts from about 1 wt % to about 20 wt %, preferably from about 5 wt % to about 15 wt % of the film.

Methods for preparing films according to the invention are capable of encapsulating the oil ingredients within the film-forming matrix and maintaining the integrity of the film, even when the film contains oils in amounts of 10 wt % or more.

In certain methods for preparing films according to the invention, the film-forming ingredients are mixed and hydrated with water separately from the water-soluble ingredients, which are mixed in aqueous solution separately from the organic ingredients and surfactants. In these methods, the final formulation is preferably produced by mixing the film-forming phase with the aqueous phase, then mixing in the organic phase, which includes surfactants, such as Polysorbate 80 and Atmos 300. This mass is mixed until emulsified. In other embodiments, the aqueous and film forming phases are combined into a single phase by dissolving the water soluble ingredients in the water and then adding the gums to hydrate. The organic phase is then added to this single aqueous phase.

The resulting formulation is cast on a suitable substrate and dried to form a film. The film is preferably air-dried or dried under warm air and cut to a desired dimension, packaged and stored. The film can contain from about 0.1% to about 10 wt % moisture, preferably from about 3% to about 8 wt % moisture, even more preferably from about 4 to about 7 wt % moisture.

The film-forming phase can include pullulan and stabilizing agents such as xanthan gum, locust bean gum and carrageenan. These ingredients are mixed and then hydrated in water for about 30 to about 48 hours to form a gel. The water is preferably heated to a temperature of about 25 to about 45° C. to promote hydration. The amount of water is about 40 to 80% of the gel. The resulting hydrated gel is then chilled to a temperature of about 20 to about 30° C. for about 1 to about 48 hours. The water is preferably deionized.

In preferred embodiments, the aqueous phase includes water heated to a temperature of about 60 to 90° C., preferably 70 to 80° C., and ingredients such as ouabain, k-strophanthin or mixtures thereof, optionally amorphous cyclodextrin, coloring agent, preservative and sweetener. The water is preferably deionized and the amount of water used is about 5 to about 80 wt % of the final gel mixture.

The water soluble digitalis glycoside is complexed to the optional amorphous cyclodextrin without separating the complexed water soluble digitalis glycoside from uncomplexed water soluble digitalis glycoside and counter ion salts. The preferred optional amorphous cyclodextrins are hydroxypropyl 3-cyclodextrin and hydroxypropyl γ-cyclodextrin.

The amount of the water soluble digitalis glycoside complexed to the optional amorphous cyclodextrin is in the range from about 25 to about 99% by weight of the water soluble digitalis glycoside/amorphous cyclodextrin complex (hereinafter referred to as the “water soluble digitalis glycoside/amorphous cyclodextrin complex” or “complex”). More preferably, the amount of the water soluble digitalis glycoside complexed to the amorphous cyclodextrin is in the range from about 50 to about 99% by weight of the water soluble digitalis glycoside/amorphous cyclodextrin complex. Most preferably, the amount of the water soluble digitalis glycoside complexed to the amorphous cyclodextrin is in the range from about 70 to about 99% by weight of the water soluble digitalis glycoside/amorphous cyclodextrin complex.

The amount of complex in the formulation is adjusted to deliver a predetermined dose of the pharmaceutically active agent over a predetermined period of time.

For example, a preferred ouabain film of the invention is administered at one dose every 8 hours to deliver a pharmaceutically effective amount of ouabain over a period of approximately 8 hours to a subject in need of such administration. A typical adult dose of a film of the invention measuring 1″×1.25″ (2.54 cm×3.18 cm) weighs about 60 to about 190 mg and contains about 2 to about 15 mg of ouabain.

In a particularly preferred embodiment of the invention, pullulan is present in the film in an amount of about 2 to about 6 mg/cm², ouabain is present in the film in an amount of about 0.2 to about 0.8 mg/cm² and optional amorphous cyclodextrin is present in said film in an amount of about 0.6 to about 2.5 mg/cm².

In embodiments, a certain percentage of the films disclosed herein will contain water soluble digitalis glycoside/amorphous cyclodextrin complexes and the remaining will be in the uncomplexed form.

In embodiments, it is possible to hydrate the film-forming ingredients and combine all of the ingredients without heating. This method comprises dissolving the water-soluble ingredients in water to form an aqueous mixture; mixing the film-forming ingredients in powder form to form a powder mixture; adding the powder mixture to the aqueous mixture to form a hydrated polymer gel; stirring the hydrated polymer at room temperature for about 30 minutes to about 48 hours; mixing the cooling agent, menthol and any other oils to form an oil mixture; adding the oil mixture to the hydrated polymer gel and mixing until uniform; deaerating the film until air bubbles are removed, casting the uniform mixture on a suitable substrate; and drying the cast mixture to form a film. This method hydrates the film-forming ingredients without heating the water, which can reduce energy costs in the manufacturing process and undesirable losses of volatile ingredients to evaporation. Further, mixing the oils in two steps minimizes the amount of flavor lost.

While not wishing to be bound by any theories, it is believed that the film-forming ingredients can be hydrated and mixed without heating due to an ionic effect known as the Donnan equilibrium. Hydrating the film-forming agents in the presence of electrolytes in solution effectively lowers the viscosity of the polymer gel being formed, thus increasing the efficiency of the hydrating process. The water-soluble ingredients of the formulation provide the electrolytes, which are dissolved in the hydration solution prior to addition of the film-forming ingredients. High-shear mixing also accelerates hydration, which delumps the powders, providing greater surface area for water contact. In addition, local heating effects, generated in the shear regions, provide energy for hydration without substantially raising the temperature of the mass.

### EXAMPLES

The invention will be illustrated in more detail with reference to the following Examples, but it should be understood that the present invention is not deemed to be limited thereto.

### Example 1

TABLE 1
Thin Film Composition containing 6 mg Ouabain in a Strip
Ouabain Thin Film Composition Jun. 29, 2013
Batch Size (gm) 100.0

| | | | | | Ouabain Overage (%) | | | |
|---|---|---|---|---|---|---|---|---|
| | | | | | 5 | 80 | | |
| | | | | | Without | Strip | | Amount |
| | Name of Ingredient | | Percent | Adj | Solvent | mg | | Batch (gm) |
| 1 | Ouabain 8H2O | | 3.3000 | 3.4650 | | | | 3.465 |
| | Ouabain | | 2.6474 | 2.7798 | 7.5224 | 6.0179 | | 2.780 |
| | 8H2O | | 0.6526 | 0.6852 | | | | 0.685 |
| 2 | Xanthan Gum | | 0.0600 | 0.0600 | 0.1624 | 0.1299 | | 0.060 |
| 3 | Locust Bean Gum | | 0.0700 | 0.0700 | 0.1894 | 0.1515 | | 0.070 |
| 2 | Carrageenan | | 0.3000 | 0.3000 | 0.8118 | 0.6495 | | 0.300 |
| 2 | Pullulan | | 16.0000 | 16.0000 | 43.2978 | 34.6382 | | 16.000 |
| 3 | Potassium Sorbate | | 0.0600 | 0.0600 | 0.1624 | 0.1299 | | 0.060 |
| 4 | Acesulfame Potassium Salt | | 0.5000 | 0.5000 | 1.3531 | 1.0824 | | 0.500 |
| 6 | Aspartame NF | | 1.4000 | 1.4000 | 3.7886 | 3.0308 | | 1.400 |
| 7 | Physcool | | 0.1000 | 0.1000 | 0.2706 | 0.2165 | | 0.100 |
| 8 | Menthol | | 1.0000 | 1.0000 | 2.7061 | 2.1649 | | 1.000 |
| 9 | Citric Acid | | 0.0710 | 0.0710 | 0.1921 | 0.1537 | | 0.071 |
| 10 | Cherry Flavor (Givudan) | | 0.1500 | 0.1500 | 0.4059 | 0.3247 | | 0.150 |
| 11 | Mono ammonium glycyrrhizinate | | 0.0100 | 0.0100 | 0.0271 | 0.0216 | | 0.010 |
| 12 | Polysorbate 80 NF | | 0.3500 | 0.3500 | 0.9471 | 0.7577 | | 0.350 |
| 13 | Atmos 300 | | 0.3500 | 0.3500 | 0.9471 | 0.7577 | | 0.350 |
| 14 | Propylene Glycol | | 3.0000 | 3.0000 | 8.1183 | 6.4947 | | 3.000 |
| 15 | Olive Oil | | 0.5000 | 0.5000 | 1.3531 | 1.0824 | | 0.500 |
| 16 | Titanium Dioxide | | 0.2500 | 0.2500 | 0.6765 | 0.5412 | | 0.250 |
| 17 | Hydroxypropyl γ-Cyclodextrin | | 10.0000 | 10.0000 | 27.0611 | 21.6489 | | 10.000 |
| 18 | FD&C red 40 | | 0.0026 | 0.0026 | 0.0070 | 0.0056 | | 0.003 |
| 19 | Purified Water | | 62.5264 | 62.3614 | | | | 62.361 |
| | TOTAL WITHOUT SOLVENT | | | 36.9534 | 100.0000 | 80.0000 | | 36.953 |
| | Total Without Active | | 97.353 | 97.2202 | | | | |
| | TOTAL WITH ACTIVE | | 100.000 | 100.000 | | | | 100.000 |
| | Total # of Strips | 462 | | Ouabain | | 584.652 | 6.00 | |
| | On the basis of Strips | 6.02 mg | | Ouabain 8H2O | | 728.77 | 7.48 | |

The ingredients listed in Table 1 were combined to provide a comparative example of an ouabain film containing 6 mg per strip in accordance with the following procedure:

A. The water was heated to 50-70° C. The ouabain and hydroxypropyl γ-cyclodextrin were added and dissolved with stirring. Then the potassium sorbate and sweeteners were dissolved in the water with mixing. The titanium dioxide was then added with further mixing to form Preparation A.
B. The film-forming ingredients (e.g., xanthan gum, locust bean gum, carrageenan and pullulan) were mixed in a separate container to form Preparation B.
C. Preparation B was slowly added to Preparation A with rapid mixing, followed by overnight mixing at a reduced rate to provide Preparation C.
D. The glycerin and olive oil were combined in a separate container and then the menthol and monoammonium glycyrrhizinate (MAG) were dissolved therein by heating to 45° C. to form Preparation D.
E. Preparation D was added to Preparation C with thorough mixing and then the flavor agents were added with continued mixing to provide Preparation E.
F. The pH was adjusted as necessary to 6.0-7.0 using 10% citric acid solution to provide Preparation F (Example 1).
Preparation F was poured on a mold and cast to form a film of a desired thickness at room temperature. The film was dried under warm air and cut to a desired dimension (dictated by, e.g., dosage and mouth feel) for taste testing. The film was segmented into 1″×1.25″ (2.54 cm×3.18 cm) dosage units, each of which had a thickness of 0.01±0.002 in (0.25±0.05 mm) and a weight of 80±3 mg.
A placebo film without the ouabain was also prepared in accordance with the foregoing to facilitate evaluation of, e.g., the taste and appearance of the active film.

TABLE 2
Thin Film Composition containing 3 mg Ouabain in a Strip
Ouabain Thin Film Composition Jun. 29, 2013
Batch Size (gm) 100.0

| | | | | | Ouabain Overage (%) | | | |
|---|---|---|---|---|---|---|---|---|
| | | | | | 5 | 80 | | |
| | | | | | Without | Strip | | Amount |
| | Name of Ingredient | | Percent | Adj | Solvent | mg | | Batch (gm) |
| 1 | Ouabain 8H2O | | 1.3600 | 1.4280 | | | | 1.428 |
| | Ouabain | | 1.0911 | 1.1456 | 3.7785 | 3.0228 | | 1.146 |
| | 8H2O | | 0.2689 | 0.2824 | | | | 0.282 |
| 2 | Xanthan Gum | | 0.0600 | 0.0600 | 0.1979 | 0.1583 | | 0.060 |
| 3 | Locust Bean Gum | | 0.0700 | 0.0700 | 0.2309 | 0.1847 | | 0.070 |
| 2 | Carrageenan | | 0.3000 | 0.3000 | 0.9895 | 0.7916 | | 0.300 |
| 2 | Pullulan | | 16.0000 | 16.0000 | 52.7718 | 42.2175 | | 16.000 |
| 3 | Potassium Sorbate | | 0.0600 | 0.0600 | 0.1979 | 0.1583 | | 0.060 |
| 4 | Acesulfame Potassium Salt | | 0.5000 | 0.5000 | 1.6491 | 1.3193 | | 0.500 |
| 6 | Aspartame NF | | 1.4000 | 1.4000 | 4.6175 | 3.6940 | | 1.400 |
| 7 | Physcool | | 0.1000 | 0.1000 | 0.3298 | 0.2639 | | 0.100 |
| 8 | Menthol | | 1.0000 | 1.0000 | 3.2982 | 2.6386 | | 1.000 |
| 9 | Citric Acid | | 0.0710 | 0.0710 | 0.2342 | 0.1873 | | 0.071 |
| 10 | Cherry Flavor (Givudan) | | 0.1500 | 0.1500 | 0.4947 | 0.3958 | | 0.150 |
| 11 | Mono ammonium glycyrrhizinate | | 0.0100 | 0.0100 | 0.0330 | 0.0264 | | 0.010 |
| 12 | Polysorbate 80 NF | | 0.3500 | 0.3500 | 1.1544 | 0.9235 | | 0.350 |
| 13 | Atmos 300 | | 0.3500 | 0.3500 | 0.1544 | 0.9235 | | 0.350 |
| 14 | Propylene Glycol | | 3.0000 | 3.0000 | 9.8947 | 7.9158 | | 3.000 |
| 15 | Olive Oil | | 0.5000 | 0.5000 | 1.6491 | 1.3193 | | 0.500 |
| 16 | Titanium Dioxide | | 0.2500 | 0.2500 | 0.8246 | 0.6596 | | 0.250 |
| 17 | Hydroxypropyl γ-Cyclodextrin | | 5.0000 | 5.0000 | 16.4912 | 13.1930 | | 5.000 |
| 18 | FD&C red 40 | | 0.0026 | 0.0026 | 0.0086 | 0.0069 | | 0.003 |
| 19 | Purified Water | | 69.4664 | 69.3984 | | | | 69.398 |
| | TOTAL WITHOUT SOLVENT | | | 30.3192 | 100.0000 | 80.0000 | | 30.319 |
| | Total Without Active | | 98.909 | 98.8544 | | | | |
| | TOTAL WITH ACTIVE | | 100.000 | 100.000 | | | | 100.000 |
| | Total # of Strips | 379 | | Ouabain | | 584.652 | 6.00 | |
| | On the basis of Strips | 3.02 mg | | Ouabain 8H2O | | 728.77 | 7.48 | |

The ingredients listed in Table 2 were combined to provide a comparative example of an ouabain film strip containing 3 mg in accordance with procedure as described above.

### Example 2

The ingredients listed in Table 3 were combined to provide a comparative example of an ouabain film containing 6 mg per strip in accordance with the following procedure:
A. The water and ethyl alcohol were heated to 50-70° C. The ouabain was added and dissolved with stirring. Then the potassium sorbate and sweeteners were dissolved in the water with mixing. The titanium dioxide was then added with further mixing to form Preparation A.
B. The film-forming ingredients (e.g., xanthan gum, locust bean gum, carrageenan and pullulan) were mixed in a separate container to form Preparation B.
C. Preparation B was slowly added to Preparation A with rapid mixing, followed by overnight mixing at a reduced rate to provide Preparation C.
D. The glycerin and olive oil were combined in a separate container and then the menthol and monoammonium glycyrrhizinate (MAG) were dissolved therein by heating to 45° C. to form Preparation D.
E. Preparation D was added to Preparation C with thorough mixing and then the flavor agents were added with continued mixing to provide Preparation E.
F. The pH was adjusted as necessary to 6.0-7.0 using 10% citric acid solution to provide Preparation F (Examples 1-2).
Preparation F was poured on a mold and cast to form a film of a desired thickness at room temperature. The film was dried under warm air and cut to a desired dimension (dictated by, e.g., dosage and mouth feel) for taste testing. The film was segmented into 1″×1.25″ (2.54 cm×3.18 cm) dosage units, each of which had a thickness of 0.01±0.002 in (0.25±0.05 mm) and a weight of 80±3 mg.
A placebo film without the ouabain was also prepared in accordance with the foregoing to facilitate evaluation of, e.g., the taste and appearance of the active film.

The ingredients listed in Table 4 were combined to provide a comparative example of an ouabain film strip containing 3 mg in accordance with procedure as described above.

TABLE 3
Thin Film Composition containing 6 mg Ouabain in a Strip without Cyclodextrin
Ouabain Thin Film Composition Without Amorphous Cyclodextrin Jun. 29, 2013
Batch Size (gm) 100.0

| | | | | | Ouabain Overage (%) | | | |
|---|---|---|---|---|---|---|---|---|
| | | | | | 5 | 80 | | |
| | | | | | Without | Strip | | Amount |
| | Name of Ingredient | | Percent | Adj | Solvent | mg | | Batch (gm) |
| 1 | Ouabain 8H2O | | 2.3500 | 2.4675 | | | | 2.468 |
| | Ouabain | | 1.8853 | 1.9795 | 7.5690 | 6.0552 | | 1.980 |
| | 8H2O | | 0.4647 | 0.4880 | | | | 0.488 |
| 2 | Xanthan Gum | | 0.0600 | 0.0600 | 0.2294 | 0.1835 | | 0.060 |
| 3 | Locust Bean Gum | | 0.0700 | 0.0700 | 0.2677 | 0.2141 | | 0.070 |
| 2 | Carrageenan | | 0.3000 | 0.3000 | 1.1471 | 0.9177 | | 0.300 |
| 2 | Pullulan | | 16.0000 | 16.0000 | 61.1781 | 48.9425 | | 16.000 |
| 3 | Potassium Sorbate | | 0.0600 | 0.0600 | 0.2294 | 0.1835 | | 0.060 |
| 4 | Acesulfame Potassium Salt | | 0.5000 | 0.5000 | 1.9118 | 1.5295 | | 0.500 |
| 6 | Aspartame NF | | 1.4000 | 1.4000 | 5.3531 | 4.2825 | | 1.400 |
| 7 | Physcool | | 0.1000 | 0.1000 | 0.3824 | 0.3059 | | 0.100 |
| 8 | Menthol | | 1.0000 | 1.0000 | 3.8236 | 3.0589 | | 1.000 |
| 9 | Citric Acid | | 0.0710 | 0.0710 | 0.2715 | 0.2172 | | 0.071 |
| 10 | Cherry Flavor (Givudan) | | 0.1500 | 0.1500 | 0.5735 | 0.4588 | | 0.150 |
| 11 | Mono ammonium glycyrrhizinate | | 0.0100 | 0.0100 | 0.0382 | 0.0306 | | 0.010 |
| 12 | Polysorbate 80 NF | | 0.3500 | 0.3500 | 1.3383 | 1.0706 | | 0.350 |
| 13 | Atmos 300 | | 0.3500 | 0.3500 | 1.3383 | 1.0706 | | 0.350 |
| 14 | Propylene Glycol | | 3.0000 | 3.0000 | 11.4709 | 9.1767 | | 3.000 |
| 15 | Olive Oil | | 0.5000 | 0.5000 | 1.9118 | 1.5295 | | 0.500 |
| 16 | Titanium Dioxide | | 0.2500 | 0.2500 | 0.9559 | 0.7647 | | 0.250 |
| 17 | Ethyl Alcohol | | 10.0000 | 10.0000 | | 0.0000 | | 10.000 |
| 18 | FD&C red 40 | | 0.0026 | 0.0026 | 0.0099 | 0.0080 | | 0.003 |
| 19 | Purified Water | | 63.4764 | 63.3589 | | | | 63.359 |
| | TOTAL WITHOUT SOLVENT | | | 26.1531 | 100.0000 | 80.0000 | | 26.153 |
| | Total Without Active | | 98.115 | 98.0205 | | | | |
| | TOTAL WITH ACTIVE | | 100.000 | 100.000 | | | | 100.000 |
| | Total # of Strips | 327 | | Ouabain | | 584.652 | 6.00 | |
| | On the basis of Strips | 6.06 mg | | Ouabain 8H2O | | 728.77 | 7.48 | |

TABLE 4
Thin Film Composition containing 3 mg Ouabain in a Strip
Ouabain Thin Film Composition Without Amorphous Cyclodextrin Jun. 29, 2013
Batch Size (gm) 100.0

| | | | | | Ouabain Overage (%) | | | |
|---|---|---|---|---|---|---|---|---|
| | | | | | 5 | 80 | | |
| | | | | | Without | Strip | | Amount |
| | Name of Ingredient | | Percent | Adj | Solvent | mg | | Batch (gm) |
| 1 | Ouabain 8H2O | | 1.1250 | 1.1813 | | | | 1.181 |
| | Ouabain | | 0.9025 | 0.9477 | 3.7723 | 3.0178 | | 0.948 |
| | 8H2O | | 0.2225 | 0.2336 | | | | 0.234 |
| 2 | Xanthan Gum | | 0.0600 | 0.0600 | 0.2388 | 0.1911 | | 0.060 |
| 3 | Locust Bean Gum | | 0.0700 | 0.0700 | 0.2786 | 0.2229 | | 0.070 |
| 2 | Carrageenan | | 0.3000 | 0.3000 | 1.1942 | 0.9554 | | 0.300 |
| 2 | Pullulan | | 16.0000 | 16.0000 | 63.6911 | 50.9529 | | 16.000 |
| 3 | Potassium Sorbate | | 0.0600 | 0.0600 | 0.2388 | 0.1911 | | 0.060 |
| 4 | Acesulfame Potassium Salt | | 0.5000 | 0.5000 | 1.9903 | 1.5923 | | 0.500 |
| 6 | Aspartame NF | | 1.4000 | 1.4000 | 5.5730 | 4.4584 | | 1.400 |
| 7 | Physcool | | 0.1000 | 0.1000 | 0.3981 | 0.3185 | | 0.100 |
| 8 | Menthol | | 1.0000 | 1.0000 | 3.9807 | 3.1846 | | 1.000 |
| 9 | Citric Acid | | 0.0710 | 0.0710 | 0.2826 | 0.2261 | | 0.071 |
| 10 | Cherry Flavor (Givudan) | | 0.1500 | 0.1500 | 0.5971 | 0.4777 | | 0.150 |
| 11 | Mono ammonium glycyrrhizinate | | 0.0100 | 0.0100 | 0.0398 | 0.0318 | | 0.010 |
| 12 | Polysorbate 80 NF | | 0.3500 | 0.3500 | 1.3932 | 1.1146 | | 0.350 |
| 13 | Atmos 300 | | 0.3500 | 0.3500 | 1.3932 | 1.1146 | | 0.350 |
| 14 | Propylene Glycol | | 3.0000 | 3.0000 | 11.9421 | 9.5537 | | 3.000 |
| 15 | Olive Oil | | 0.5000 | 0.5000 | 1.9903 | 1.5923 | | 0.500 |
| 16 | Titanium Dioxide | | 0.2500 | 0.2500 | 0.9952 | 0.7961 | | 0.250 |
| 17 | Ethyl Alcohol | | 5.0000 | 5.0000 | | 0.0000 | | 5.000 |
| 18 | FD&C red 40 | | 0.0026 | 0.0026 | 0.0103 | 0.0083 | | 0.003 |
| 19 | Purified Water | | 69.7014 | 69.6452 | | | | 69.645 |
| | TOTAL WITHOUT SOLVENT | | | 25.1213 | 100.0000 | 80.0000 | | 25.121 |
| | Total Without Active | | 99.097 | 99.0523 | | | | |
| | TOTAL WITH ACTIVE | | 100.000 | 100.000 | | | | 100.000 |
| | Total # of Strips | 314 | | Ouabain | | 584.652 | 6.00 | |
| | On the basis of Strips | 3.02 mg | | Ouabain 8H2O | | 728.77 | 7.48 | |

What is claimed is:

1. An orally consumable rapidly dissolving solid film comprising: at least one water soluble polymer, and at least one water soluble digitalis glycoside and optionally at least one amorphous cyclodextrin; and wherein the ratio of the at least one water soluble digitalis glycoside to the optionally at least one amorphous cyclodextrin is about 1:1 to about 1:10 and wherein said orally consumable film is adapted to adhere to and dissolve in the mouth of a subject afflicted with heart disease.

2. The orally consumable rapidly dissolving solid film according to claim 1, wherein said water soluble polymer is selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

3. The orally consumable rapidly dissolving solid film according to claim 2, wherein said water soluble polymer is pullulan.

4. The orally consumable rapidly dissolving solid film according to claim 1, wherein said water soluble digitalis glycoside is selected from the group consisting of ouabain, k-strophanthin and mixtures thereof.

5. The orally consumable rapidly dissolving solid film according to claim 1 wherein the water soluble digitalis glycoside provides from about 3 wt % to about 20 wt % of said optional amorphous cyclodextrin.

6. The orally consumable rapidly dissolving solid film according to claim 5, wherein said optional amorphous cyclodextrin is selected from hydroxypropyl, hydroxyethyl, glucosyl, maltosyl, maltotriosyl, carboxyamidomethyl, carboxymethyl, hydroxypropyl, sulfobutylether and diethylamino derivatives of β- and γ-cyclodextrin.

7. The orally consumable rapidly dissolving solid film according to claim 5, wherein said water soluble polymer is pullulan, said water soluble digitalis glycoside is ouabain, and said optional amorphous cyclodextrin is hydroxypropyl β-cyclodextrin or hydroxypropyl γ-cyclodextrin.

8. The orally consumable rapidly dissolving solid film according to claim 7, comprising pullulan in an amount of about 30 to about 80 wt % of said film, ouabain in an amount of about 3 to about 20 wt % of said film, and optionally amorphous cyclodextrin in an amount of about 5 to about 40 wt % of said film.

9. A method for preparing the orally consumable rapidly dissolving solid film of claim 1, said method comprising: dissolving the water-soluble polymer in water to provide an aqueous solution; mixing water soluble film former and stabilizing agent to provide a solid-film forming mixture; combining said solid-film forming mixture and said aqueous solution to provide a hydrated polymer gel; mixing oils to form an oil mixture; admixing said oil mixture and said hydrated polymer gel to provide a uniform gel, said uniform gel comprising said water soluble digitalis glycoside and said optionally at least one amorphous cyclodextrin; casting the uniform gel on a substrate; and drying the cast gel to provide said solid film.

10. The method of claim 9 wherein said aqueous solution comprises both said water soluble digitalis glycoside and said optional amorphous cyclodextrin.

11. The method of claim 9, wherein said water soluble digitalis glycoside is complexed to said optional amorphous cyclodextrin without separating complexed water soluble digitalis glycoside from uncomplexed water soluble digitalis glycoside and counter ion salts.

12. An orally consumable solid film comprising a water soluble polymer, a water soluble digitalis glycoside and optionally an amorphous cyclodextrin wherein said amorphous cyclodextrin is present at a weight ratio to said water soluble digitalis glycoside of about 5:1 to about 10:1 and said orally consumable film is adapted to adhere to and dissolve in the mouth of a subject afflicted with heart disease.

13. The orally consumable solid film according to claim 8, wherein pullulan is present in the film in an amount of about 2 to about 6 mg/cm², ouabain is present in the film in an amount of about 0.2 to about 0.8 mg/cm² and optional amorphous cyclodextrin is present in said film in an amount of about 0.6 to about 2.5 mg/cm².

14. The orally consumable solid film according to claim 8 or 13, further comprising: about 0.01 to about 5 w % of at least one stabilizing agent; about 0.001 to about 0.1 wt % of at least one of at least one coloring agent; about 0.01 to about 70 wt % water; about 0.1 to about 15 wt % of at least one sweetening agent; about 0.1 to about 15 w % of at least one flavoring agent; about 0.1 to about 4 wt % of at least one cooling agent; about 0.1 to about 5 wt % of at least one surfactant; about 0.1 to about 12 wt % of a triglyceride; about 0.001 to about 5 wt % of a preservative; about 0.01 to about 5 wt % of a polyethylene oxide compound; and about 1 to about 20 wt % of propylene glycol.

15. The orally consumable solid film according to claim 1 wherein the water soluble digitalis glycoside comprises ouabain.

16. The orally consumable solid film according to claim 1 wherein the water soluble digitalis glycoside comprises k-strophanthin.

17. The orally consumable solid film according to claim 1 wherein the water soluble digitalis glycoside comprises a mixture of ouabain and k-strophanthin.

18. The orally consumable solid film according to claim 2 wherein said water soluble polymer comprises polyvinyl alcohol.

19. The orally consumable solid film according to claim 2 wherein said water soluble polymer comprises hydroxypropyl cellulose.

20. The orally consumable solid film according to claim 1, wherein said film has a thickness of 0.01±0.002 in.

21. The orally consumable solid film according to claim 1, wherein said film contains about 0.1% to about 10 wt % moisture, preferably about 3% to about 8 wt % moisture and most preferably about 4% to about 7 wt % moisture.

22. A method of treating heart disease in a subject wherein the said method comprises administering an orally consumable solid film of claim 1 to the subject by mouth.

23. The method of claim 22, wherein the heart disease is selected from the group consisting of myocardial infarction, congestive heart insufficiency without pronounced hypertrophy, coronary sclerosis, cardiogenic hypertension, cardiac asthma, exercise-induced cardiac insufficiency, angina pectoris and arrhythmias, including those that occur on treatment with digitalis.
